(19) United States
(12) Patent Application Publication
(10) Pub. No.: US 2023/0056788 A1
(43) Pub. Date: Feb. 23, 2023

(54) METHODS AND COMPOSITIONS FOR THE INHIBITION OF HEPATITIS B AND HEPATITIS D VIRUS INFECTIONS

(71) Applicant: REPLICOR INC., Montreal (CA)

(72) Inventors: Andrew VAILLANT, Roxboro (CA); Richard BOULON, Montréal (CA); Matthieu BLANCHET, Montreal (CA); Patrick LABONTE, Laval (CA)

(21) Appl. No.: 17/792,746

(22) PCT Filed: Feb. 18, 2021
(86) PCT No.: PCT/CA2021/050176

§ 371 Date: Jul. 14, 2022

Related U.S. Application Data
(60) Provisional application No. 62/979,442, filed on Feb. 21, 2020.

(51) Int. Cl. C12N 15/113; A61P 31/20

(52) U.S. Cl. CPC C12N 15/1131; A61P 31/20; C12N 2310/14; C12N 2310/315

(57) ABSTRACT

The present disclosure relates to methods for the inhibition of proteins involved in the assembly and or secretion of HBV SVP by inhibiting the activity of casein kinase 1 isoform delta, DNAJB12, and/or microtubule-actin crosslinking factor 1.

### CROSS-REFERENCE TO RELATED APPLICATION

The present application is claiming priority from U.S. Provisional Application No. 62/979,442 filed Feb. 21, 2020, and U.S. Provisional Application No. 63/078,939 filed Sep. 16, 2020 the content of which are hereby incorporated by reference in their entirety.

### TECHNICAL FIELD

The present disclosure relates to compositions and methods for the inhibition hepatitis B (HBV) or hepatitis D (HDV) virus infection by targeting a variety of novel proteins involved in the assembly and secretion of HBV subviral particles (SVP).

### BACKGROUND ART

Hepatitis B virus (HBV) is an enveloped virus of the family hepadnaviridae. This virus is the cause of the largest known pandemic viral infection, having affected more than 2 billion people worldwide and leaving more than 300 million of these individuals with chronic liver infection. Annually, there are approximately 870,000 deaths attributable to the effects of HBV infection. To meet the need for treatment of this disease, several drugs have been approved for the treatment of HBV which include immunotherapies such as pegylated interferons and thymosin alpha 1, and a variety of nucleos(t)ide analog inhibitors of the HBV reverse transcriptase such as entecavir and tenofovir disoproxil fumarate. However, while these drugs can suppress the replication of the virus in the liver, they have little to no effect on the production of HBV subviral particles (SVP). These SVP provide the bulk of the circulating hepatitis B surface antigen protein (HBsAg) and can be produced independently from replication using integrated HBV DNA as the genetic source. Circulating HBsAg is the most abundant circulating viral antigen and has important functions in inhibiting the immune response to HBV infection. The sustained clearance of HBsAg in the absence of treatment is widely acknowledged as the best marker for true restoration of immune control of the infection and restoration of normal liver function (functional cure) and is the current goal for new therapies in development. As such, there is still a need for a more effective treatment for HBV which can effectively target the production of SVP.

The only current therapy in development which has demonstrated an ability to achieve a high rate of clearance of HBsAg during therapy and functional cure of HBV and HDV infection (persistent clearance of HDV RNA in the absence of therapy) are nucleic acid polymers (NAPs). NAPs selectively interfere with the assembly and secretion of SVP without affecting the production or secretion of virions or other viral antigens such as hepatitis E antigen. However, to date little is known either about the molecular mechanisms underlying the assembly and secretion of SVP or of the targets for NAPs driving their antiviral effects in HBV and HDV infection.

HDV is a defective virus considered a satellite infection of HBV that requires HBsAg derived from the HBV genome to form its envelope. The HBsAg isoform composition of HDV is the same as for HBV SVP, indicating that similar machinery is used for the assembly and or secretion of HBV SVP and HDV. It would thus be desirable to be provided with more effective treatment for HBV and or HDV infection by the inhibition of targets involved in the assembly and or secretion of HBV SVP and HDV.

### SUMMARY

In accordance with the present disclosure there is now provided a method for the inhibition of HBV infection or HBV/HDV co-infection, the method comprising administering to a patient in need of such treatment a pharmacologically acceptable small molecule inhibiting the function of one or more of the following proteins: casein kinase I isoform delta, DNAJB12, or microtubule-actin crosslinking factor 1.

It is also provided a method for the inhibition of HBV infection or HBV/HDV co-infection, the method comprising administering to a patient in need of such treatment a pharmacologically acceptable antisense oligonucleotide complementary to any part of the mRNA of one or more of the following proteins: casein kinase I isoform delta, DNAJB12, or microtubule-actin crosslinking factor 1.

It is also provided a method for the inhibition of HBV infection or HBV/HDV co-infection, the method comprising administering to a patient in need of such treatment a pharmacologically acceptable synthetic interfering RNA (siRNA) complementary to any part of the mRNA of one or more of the following proteins: casein kinase I isoform delta, DNAJB12, or microtubule-actin crosslinking factor 1.

It is also provided a method for the inhibition of HBV infection or HBV/HDV co-infection, the method comprising administering to a patient in need of such treatment a pharmacologically acceptable CRISPR-associated endonuclease and a guide RNA (gRNA) complementary to any part of the mRNA of one or more of the following proteins: casein kinase 1 isoform delta, DNAJB12 or microtubule-actin crosslinking factor 1.

It is also provided a composition comprising a pharmacologically acceptable small molecule inhibiting the function of one or more of the following proteins: casein kinase I isoform delta, DNAJB12 or microtubule-actin crosslinking factor 1.

It is also provided a composition comprising a pharmacologically acceptable antisense oligonucleotide complementary to any part of the mRNA of one or more of the following proteins: casein kinase I isoform delta, DNAJB12 or microtubule-actin crosslinking factor 1.

It is also provided a composition comprising a pharmacologically acceptable synthetic interfering RNA (siRNA) complementary to any part of the mRNA of one or more of the following proteins: casein kinase I isoform delta, DNAJB12 or microtubule-actin crosslinking factor 1.

It is also provided a composition for the inhibition of HBV infection or HBV/HDV co-infection, the composition comprising a pharmacologically acceptable CRISPR-associated endonuclease and a guide RNA (gRNA) complementary to any part of the mRNA of one or more of the following proteins: casein kinase 1 isoform delta, DNAJB12 or microtubule-actin crosslinking factor 1.

In an embodiment, the small molecule is an oligonucleotide.

In a further embodiment, the oligonucleotide is an antisense oligonucleotide, a synthetic interfering RNA or CRISPR-associated endonuclease and a guide RNA (gRNA) complementary to any part of the mRNA for casein kinase 1 isoform delta, DNAJB12 or microtubule-actin crosslinking factor 1.

In an embodiment, the small molecule is an antisense oligonucleotide with the sequence as set forth in SEQ ID NO: 12, 13 or 17.

In an alternative embodiment, the small molecule is a synthetic interfering RNA with the sequence as set forth in SEQ ID NO: 5, 6, 10, 12, 13 or 17.

In an additional embodiment, the small molecule is CRISPR-Cas9 with a guide RNA comprising the sequence as set forth in SEQ ID NO: 5, 6, 10, 12, 13 or 17.

In an additional embodiment, the oligonucleotide comprises a modified nucleobase.

In a supplemental embodiment, the oligonucleotide is single stranded or double stranded.

In another embodiment, the oligonucleotide is a Speigelmer or an aptamer.

In an embodiment, the oligonucleotide comprises at least one modification in the phosphodiester linkage, on the sugar, and on the base.

In another embodiment, the oligonucleotide comprises at least one of a phosphorothioate linkage, a phosphorodithioate linkages, a 2′-O-methyl modification, a 2′-amino modification, a 2′-halo modification, an acyclic nucleotide analog, a 3′- and/or 5′-cap, 5′methylation of the cytosine base, and a 4′thioation of the uracil base.

In an additional embodiment, the composition encompassed herein further comprises at least one nucleic acid polymer consisting of SEQ ID NO:1 and SEQ ID NO: 4.

In another embodiment, the methods and compositions defined herein inhibit the assembly and/or secretion of HBV subviral particles (SVP).

It is further provided a method of inhibiting the assembly and/or secretion of HBV subviral particles (SVP) in a patient comprising administering any of the compositions as defined herein.

It is further provided the use of a the composition as defined herein for inhibiting the assembly and/or secretion of HBV subviral particles (SVP) in a patient.

It is further provided a method for the treatment of hepatitis B or hepatitis D infection comprising administering to a subject in need of treatment an effective amount of a pharmaceutically acceptable small molecule inhibiting the activity of casein kinase 1 isoform delta, DNAJB12, and microtubule-actin crosslinking factor 1.

It is also provided the use the composition as defined herein for inhibiting the assembly and/or secretion of HBV subviral particles (SVP) in a patient.

### BRIEF DESCRIPTION OF THE DRAWINGS

Reference will now be made to the accompanying drawings, and in which:

FIG. 1 illustrates the volcano plot for selective protein target interactions with REP 2139 versus REP 2147. Each dot represents a protein which interacts with REP 2139 or REP 2147. Lighter dots indicate those proteins with selectivity ratio>2 (REP 2139: REP 2147) with p<0.05. Darker dots indicate the 6 candidates identified.

FIG. 2 illustrates the volcano plot for the selective protein target interactions with REP 2139 versus REP 2179. Each dot represents a protein which interacts with REP 2139 or REP 2179. Lighter dots indicate those proteins with selectivity ratio>2 (REP 2139: REP 2179) with p<0.05. Darker dots indicate the 6 candidates identified.

FIG. 3 illustrates the inhibition of HBsAg secretion in HepG2.2.15 cells in the presence of shRNA-mediated knockdown of targets of NAP interaction as described in Example 1. Mock, no shRNA; CSNK1D, casein kinase 1 isoform delta; CopE, coatomer subunit epsilon; CSNK1A1, casein kinase 1 isoform alpha 1; TBL2, transducing beta-like protein 2; MACF1, microtubule-actin crosslinking factor 1; CopA, coatomer subunit alpha.

### DETAILED DESCRIPTION

In accordance with the present disclosure there is now provided a method for the inhibition of HBV infection or HBV/HDV co-infection by using a small molecule inhibiting the function of one or more of the following proteins: casein kinase I isoform delta, DNAJB12, or microtubule-actin crosslinking factor 1.

HBV afflicts 300 million individuals worldwide and causes an estimated 870,000 deaths each year from complications arising from HBV infection. While several antiviral treatments are approved for use, none of these is able to elicit a therapeutically effective immune response capable of providing durable control of infection except in a small fraction of patients undergoing treatment.

HBV infection results in the production of a variety of different particles including: 1) the infectious HBV mature virion (or Dane particle) which includes a viral capsid assembled from the HBV core antigen protein (HBcAg) and is covered by the HBV surface antigen (HBsAg) and 2) non-infectious filaments, a result of defective virion/capsid interaction and 3) non-infectious spherical subviral particles (or SVPs) which are high density lipoprotein-like particles comprised of lipids, cholesterol, cholesterol esters and HBV surface antigen (HBsAg). For each viral particle produced, 10,000-100,000 SVPs are released into the blood. As such SVPs (and the HBsAg protein they carry) represent the overwhelming majority of viral protein in the blood. HBV infected cells also secrete a soluble proteolytic product of the pre-core protein called the HBV e-antigen (HBeAg).

HDV is a defective virus and uses HBsAg derived from co-existent HBV infection to form its viral envelope (Taylor, 2006, Virology, 344: 71-76) and as such, HDV infection can only occur in subjects with concomitant HBV infection. While the incidence of HDV co-infection in asymptomatic HBV carriers and chronic HBV-related liver disease is low in countries with a low incidence of HBV infection, it is a significant complication in HBV-infected subjects in countries with a high incidence of HBV infection and can increase the rate of progression of liver disease to liver cirrhosis. The unmet medical need in HBV infection is even more pressing in HBV/HDV co-infected subjects as there is no specific approved agent that directly targets the HDV virus and patient response even to combination therapy with approved agents for HBV treatment is poorer than in patients with HBV mono-infection.

The current approved treatments for HBV include interferon-α or thymosin α1-based immunotherapies and the suppression of viral production by inhibition of the HBV polymerase by nucleoside/nucleotide analogs. HBV polymerase inhibitors are effective in reducing the production of infectious virions but have little to no effect in reducing HBsAg or only very slowly reduce HBsAg with long term treatment in a limited number of patients (Fung et al., 2011, Am. J. Gasteroenterol., 106: 1766-1773; Reijnders et al., 2011, J. Hepatol., 54: 449-454). The primary effect of HBV polymerase inhibitors is to block the transformation of pre-genomic viral mRNA into partially double stranded DNA, which is present in infectious virions. Interferon based immunotherapy can achieve a reduction of infectious virus and removal of HBsAg from the blood but only in a small percentage of treated subjects.

HBsAg plays a key role in HBV infection and HBV/HDV co-infection. Aside from its role as an essential structural component for virion formation, HBsAg is also released in large amounts into the blood of infected subjects in the form of subviral particles (SVPs), which lack the viral capsid and genome and which appear to function primarily to deliver HBsAg into the blood. SVPs are secreted from infected cells in 10,000-100,000 fold excess over Dane particles which allows SVPs to effectively sequester HBsAg antibodies (anti-HBs) so that HBV or HDV virus in the blood can escape recognition by adaptive immunity. Several studies have also suggested that HBsAg directly blocks activation of adaptive and innate immune responses to HBV infection (Vaillant, ACS Infectious Diseases 2020, published online ahead of print on Dec. 10). The presence of this functionality in human HBV infection and its impact on the activity of immunotherapeutic agents and the additional applicability of these antiviral effects in HBV/HDV co-infection has been previously described in US 2014/0065102 A1, which is incorporated herein by reference in its entirety.

Another critical feature of chronic HBV infection is the establishment of stable reservoir of HBV genetic information in the nucleus of infected cells consisting of covalently closed circular DNA (cccDNA), also referred to as the HBV minichromosome, and chromosomally integrated HBV DNA. cccDNA exists in multiple copies within the nucleus and functions as the transcriptional template for the production of mRNA encoding all viral proteins and immature genomes (pre-genomic RNA) for the production of new virions. Integrated HBV DNA cannot produce pre-genomic RNA and so cannot produce virions. However, it can exist as an independent source of HBsAg (and SVP) not affected by antiviral approaches which directly target viral replication.

The term oligonucleotide (ON) refers to an oligomer or polymer of ribonucleic acid (RNA) and/or deoxyribonucleic acid (DNA). This term includes ONs composed of modified nucleobases (including 5′methylcytosine and 4′thiouracil), sugars and covalent internucleoside (backbone) linkages as well as ONs having non-naturally-occurring portions which function similarly. Such modified or substituted ONs may be preferable over native forms because of desirable properties such as, for example, reduced immunoreactivity, enhanced cellular uptake, enhanced affinity for the nucleic acid target (in the context of antisense ONs, siRNAs and shRNAs) and/or increased stability to nuclease-mediated degradation. ONs can also be double stranded. ONs also include single stranded molecules such as antisense oligonucleotides, Speigelmers and aptamers and miRNAs, as well as double stranded molecules such as small interfering RNAs (siRNAs) or small hairpin RNAs (shRNAs).

ONs can include various modifications, e.g., stabilizing modifications, and thus can include at least one modification in the phosphodiester linkage and/or on the sugar, and/or on the base. For example, the ON can include, without restriction, one or more modifications, or be fully modified so as to contain all linkages or sugars or bases with the recited modifications. Modified linkages can include phosphorothioate linkages and phosphorodithioate linkages. While modified linkages are useful, the ONs can include phosphodiester linkages. Additional useful modifications include, without restriction, modifications at the 2′-position of the sugar including 2′-O-alkyl modifications such as 2′-O-methyl modifications, 2′ O-methoxyethyl (2′ MOE), 2′-amino modifications, 2′-halo modifications such as 2′-fluoro; acyclic nucleotide analogs. Other 2′ modifications are also known in the art and can be used such as locked nucleic acids. In particular, the ON has modified linkages throughout or has every linkage modified, e.g., phosphorothioate; has a 3′-and/or 5′-cap; includes a terminal 3′-5′ linkage; the ON is or includes a concatemer consisting of two or more ON sequences joined by a linker(s). Base modifications can include 5′methylation of the cytosine base (5′ methylcytosine or in the context of a nucleotide, 5′ methylcytidine) and/or 4′thioation of the uracil base (4′thiouracil or in the context of a nucleotide, 4′thiouridine). Different chemically compatible modified linkages can be combined where the synthesis conditions are chemically compatible such as having an oligonucleotide with phosphorothioate linkages, a 2′ ribose modification (such as 2′O-methylation) and a modified base (such as 5′methylcytosine). The ON can further be completely modified with all of these different modifications (e.g. each linkage phosphorothioated, each ribose 2′ modified and each base being modified).

As encompassed herein, the term “nucleic acid polymer” or NAP is any single stranded ON which contains no sequence specific functionality, either to hybridize with a nucleic acid target or adopt a sequence specific secondary structure which results in binding to a specific protein. The biochemical activity of NAPs are not dependent on Toll-like receptor recognition of ONs, hybridization with a target nucleic acid or aptameric interaction requiring a specific secondary/tertiary ON structure derived from a specific order of nucleotides present. NAPs can include base and or linkage and or sugar modifications as described in U.S. Pat. Nos. 8,067,385, 8,008,270, 8,513,211, and 8,008,269. NAPs require phosphorothioation to have antiviral activity and a length (typically more than 20 nucleotides) to exert their antiviral effects.

Antisense ONs either singled stranded or double stranded (e.g. synthetic interfering RNA (siRNA) or small hairpin RNA (shRNA)) are designed to target a specific region of a messenger RNA (mRNA) or a micro RNA (miRNA) of interest by a specific hybridization between the antisense ON and sequence in the targeted portion of the mRNA of interest. When antisense ONs are introduced into a cell, they result in the formation of a duplex region on the mRNA or with the miRNA which directs the degradation of this specific mRNA or miRNA by RNAse H. When siRNAs is introduced into the cell (or shRNA is expressed in the cell), the antisense strand (or guide strand) is incorporated into the RISC (RNA-induced silencing complex) which uses guide-strand targeted hybridization with the complementary region on a target mRNA to effect its cleavage by the catalytic component of the RISC called Argonaute. The identification, design and optimization of antisense and siRNA are very well defined in the art and only require the sequence of the target mRNA.

CRSPR-Cas9 uses the activity of the CRISPR-associated endonuclease (Cas9 protein) and a guide RNA (gRNA) engineered to target the gene of interest. In concert, the gRNA directs the Cas9 activity to the gene of interest to splice in defective sequence, thus permanently preventing the transcription of functional mRNA. The identification, design and optimization of CRISPR-Cas9 is very well defined in the art and only requires the sequence of the target gene.

Oliognucleotide aptamers are oligonucleotides which adopt sequence-specific and selective protein interactions due to the three-dimensonal structure formed by these olognucleotides. Aptamers can be either rationally designed or selected from degenerate libraries using systematic evolution of ligands by exponential enrichment (SELEX) and the protein target of interest. Aptamers can also be built from L-ribose nucleotides which are highly resistance to nuclease degregation, also known as Speigelmers. Aptamers can also be modified as descrived above for oligonucleotides to optimize the specificity and/or strength of protein interactions and to oprtmize their pharmaceutical suitability.

The present disclosure will be more readily understood by referring to the following examples.

### Example I

### Identification of Target Interactors for NAPs

The biological basis for the antiviral activity of NAPs is the interaction of these polymers with exposed hydrophobic surfaces of amphipathic alpha helices (Vaillant, 2019, ACS Inf Dis, 10: 675-687). The current lead NAP is REP 2139 (see table 1; SEQ ID NO: 1) a fully phosphorothioated, oligonucleotide with the sequence (2′OMe adenosine,2′OMe-5-Me cytidine)₂₀. This NAP has been shown to be safe, well tolerated and potently active against infections with multiple HBV genotypes, in HBeAg positive and negative chronic infection and in HBV/HDV co-infection (Vaillant, 2019, ACS Inf Dis, 10: 675-687). The presence of the 2′OMe modification along the length of the polymer does not affect antiviral activity (Al-Mahtab et al., 2016, PLoS ONE, 11: e0156667, Roehl et al., 2017, Mol Ther Nuc Acids, 8: 1-12) but increases hydration along the long axis of the polymer, improving water solubility and reducing off target interactions. The NAP REP 2147 (SEQ ID NO: 2) is the non phosphorothioated counterpart of REP 2139 (see table 1) which is inactive. The NAP REP 2179 (SEQ ID NO: 3) is the 20mer counterpart of REP 2139 and is also inactive (Blanchet et al., 2019, Antiviral Res., 164: 97-105). These three NAPs provide biologically validated selection tools to identify the host protein targets involved in SVP assembly and or secretion.

TABLE 1
biotinylated NAPs used in target identification

| NAP | sequence | Linkage modification |
|---|---|---|
| REP 2139 | (2′OMeA, 2′OMe-5-MeC)₂₀ | Phosphorothioate |
| | SEQ ID NO: 1 | (hydrophobic, optimum |
| | | length [40mer]) |
| REP 2147 | (2′OMeA, 2′OMe-5-MeC)₂₀ | Phosphodiester |
| | SEQ ID NO: 2 | (non-hydrophobic) |
| REP 2179 | (2′OMeA, 2′OMe-5-MeC)₁₀ | Phosphorothioate |
| | SEQ ID NO: 3 | (hydrophobic, inactive |
| | | length [20mer]) |

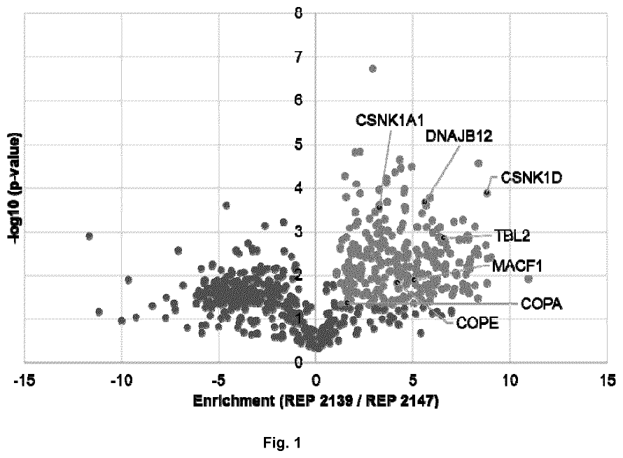

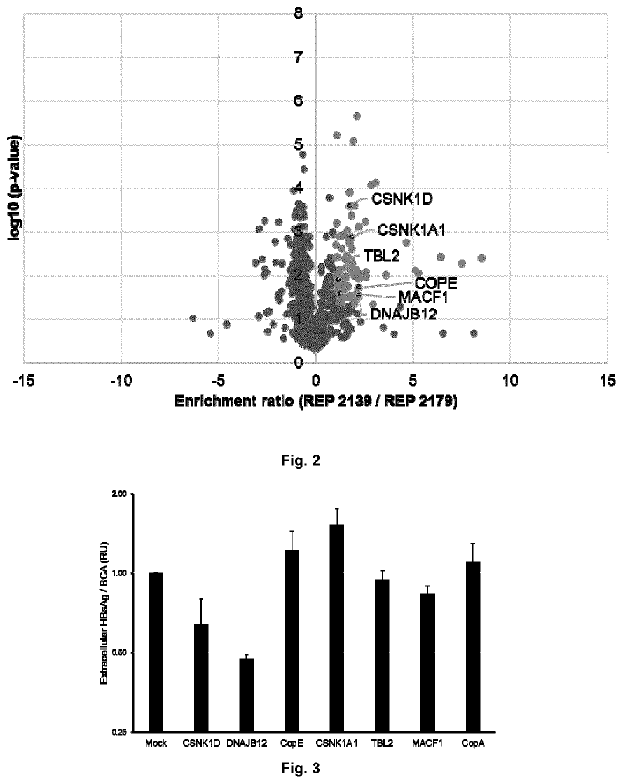

HepG2.2.15 cells are an in vitro model of HBV infection which recapitulate the production of virions and SVP and in which the biological responses of NAPs are comparable to those observed both in vivo and in human studies (Al-Mahtab et al., 2016, PLoS ONE, 11: e0156667, Bazinet et al., 2017, Lancet Gastro Hepatol, 12: 877-889; Quinet et al., 2018, Hepatol, 67: 2127-2140; Blanchet at al., 2019, Antiviral Res, 164: 97-105). Cell lysates were prepared from these cells and probed in triplicate with biotinylated REP 2139, REP 2147 and REP 2179. For each NAP, bound proteins were identified by mass spectrometry. Then a selection process was applied to these three protein subsets to identify proteins which selectively bound REP 2139 over REP 2147 and proteins with selectively bound REP 2139 over REP 2179. The volcano plots for these analyses are shown in FIGS. 1 and 2 which plot the relative enrichment ratios (x-axis) versus the statistical significance of this enrichment. This selection process identified 299 protein candidates which selectively bound REP 2139 over REP 2147 and 82 candidates which selectively bound REP 2139 over REP 2179.

From these identified proteins, final candidates were identified which demonstrated the greatest selectively for being bound by REP 2139 over REP 2147 and REP 2139 over REP 2179 and did not have previously characterized DNA/RNA binding activity. These proteins were identified as:

1. casein kinase I isoform delta (CSNK1D), involved in modulating microtubule-based vesicle transport
2. DNAJB12, an ER-resident chaperone with previously uncharacterized function
3. coatomer subunit epsilon (COPE), involved in vesicle formation and retrograde transport from the ER to the Golgi
4. casein kinase I isoform alpha (CSNK1A), involved in modulating microtubule-based vesicle transport
5. transducin beta-like protein 2 (TBL-2)_, an ER resident integral membrane protein implicated in signaling in the ER stress response
6. microtubule-actin crosslinking factor 1 (MCAF-1), a protein involved in cytoskeletal interactions at the cell periphery

All of the above protein interactions with NAPs demonstrate the same structure binding relationship as observed for antiviral activity with NAPs. This screening approach has identified for the first time novel NAP interactors which follow the structure function relationship established for NAPs in vitro and in vivo against HBV. All these targets are either involved in intracellular protein morphogenesis or transit and secretion and are candidates for the protein(s) targeted by NAPs to inhibit the morphogenesis and secretion of HBV SVP. As such, all of these novel proteins represent appropriately characterized potential therapeutic targets for the inhibition of SVP assembly and secretion. Methods and compositions for the treatment of HBV infection that involve the inhibition of the function of said proteins (using a small molecule-based approach) or reducing the production of said proteins (using antisense oligonucleotides or synthetic interfering RNA) will be equally effective against HDV infection.

### Example II

### Validation of Target Interactors for NAPs

To examine the potential role of each of the NAP interactors identified above in SVP assembly and or secretion, their expression in HepG2.2.15 cells was knocked down using an shRNA approach. An outlier protein (COPA), where NAP interaction was not size selective, was selected as a negative control.

Lentiviral constructs were used as vectors for the expression of short hairpin RNA (shRNA). The psPAX2, pMD2.G, and pRSV-REV plasmids were purchased from Addgene. Target sequences (Table 2) for the specific knockdown were identified using the MISSION® online tool (https://www.sigmaaldrich.com) and cloned into the lentivirus plasmid MISSION pLKO.1-puro. Lentiviral vectors were produced by the transfection of HEK293T cells with pLKO.1-puro derivatives, along with packaging plasmids psPAX2, pMD2.G, and pRSV-REV. The supernatants from these transfected cells were collected at day 2 post-transfection, clarified, and filtered (0.45 μm).

TABLE 2
target sequences used to design shRNA

| | | SEQ | | SEQ |
|---|---|---|---|---|
| | | ID | | ID |
| Target | Sense (5′-3′) | NO. | Antisense (5′-3′) | NO. |
| CSNK1D | AAGAGACAGAAATACGAA | 5 | TTCGTATTTCTGTCTCTT | 12 |
| DNAJB12 | CAAGGTGATGGACTGTAT | 6 | ATACAGTCCATCACCTT | 13 |
| CopE | AGCTGTTCGACGTAAAGA | 7 | TCTTTACGTCGAACAGCT | 14 |
| CSNK1A1 | AGAATTTGCGATGTACTT | 8 | AAGTACATCGCAAATTCT | 15 |
| TBL2 | TGTCATCGACATTGGCAT | 9 | AATGCCAATGTCGATGAC | 16 |
| MACF1 | CCACAACTAAAGGAATTA | 10 | TTAATTCCTTTAGTTGTG | 17 |
| CopA | GTGAGTACATTGTGGGTT | 11 | AAACCCACAATGTACTCA | 18 |

HepG2.2.15 cells were maintained in William's medium E (WME) complemented with 10% fetal bovine serum, 1% glutamine and 0.1% gentamycin at 37° C. in a humidified incubator with 5% CO2. Twenty-four hours before transfection, cells were trypsinized and seeded in 24-well plates at density of 1×10⁵ cell per wells. HepG2.2.15 target cells were inoculated with the lentiviral constructs for 16 h, in the presence of 8 μg/ml Polybrene (Sigma) and further cultured for 3 days. As controls, mock cells were transduced with lentiviral vectors containing no shRNA sequence (pLK0.1 SHC001 or SHC002). Cells were then harvested, lysed, and analyzed by RT-qPCR, proteomic quantification by BCA method and HBsAg ELISA. The HBsAg levels in cell lysates and/or in supernatants were measured by enzyme-linked immunosorbent assay (ELISA) using Genetic Systems™ HBsAg EIA 3.0 (Biorad). The quantification was conducted using a standard curve from dilution of HepG2.2.15 supernatant. All results were normalized according to total cell protein determined by BCA protein assay.

The results of these experiments identified three NAP interactors which inhibited the release of HBsAg from HepG2.2.15 cells (FIG. 3). These interactors are casein kinase 1 delta (CSNK1D), DNAJB12 and microtubule-actin crosslinking factor 1 (MCAF1). As such, methods to either directly interfere with the function of these proteins by the use of small molecules or to degrade the mRNA for these proteins by the use of antisense or siRNA or to disrupt the genes for these proteins by the use of CRISPR-Cas9 can be easily derived by any person skilled in the art. Compositions and methods for the treatment of HBV and HBV/HDV infection by antisense, RNAi or CRISPR-Cas9 can include target sequences for CSNK1, DNAJB12 or MCAF-1 as described above in Table 2 or using any other appropriate mRNA or gene sequence for these targets using methods well known and established in the art.

While the present disclosure has been described in connection with specific embodiments thereof, it will be understood that it is capable of further modifications and this application is intended to cover any variations, uses, or adaptations including such departures from the present disclosure as come within known or customary practice within the art and as may be applied to the essential features hereinbefore set forth, and as follows in the scope of the appended claims.

1. A composition for the treatment of hepatitis B or hepatitis D infection comprising a pharmaceutically acceptable small molecule inhibiting the activity of at least one of casein kinase 1 isoform delta, DNAJB12, and microtubule-actin crosslinking factor 1, and a carrier.

2. (canceled)

3. The composition of claim 1, wherein the oligonucleotide is an antisense oligonucleotide, a synthetic interfering RNA, or a CRISPR-associated endonuclease and guide RNA complementary to any part of the mRNA for casein kinase 1 isoform delta, DNAJB12, or microtubule-actin crosslinking factor 1.

4. The composition of claim 1, wherein said composition inhibiting the assembly and/or secretion of HBV subviral particles (SVP).

5. The composition of claim 1, wherein the small molecule is an antisense oligonucleotide with the sequence as set forth in SEQ ID NO: 12, 13 or 17.

6. The composition of claim 1, wherein the small molecule is a synthetic interfering RNA with the sequence as set forth in SEQ ID NO: 5, 6, 10, 12, 13 or 17.

7. The composition of claim 1, wherein the small molecule is CRISPR-Cas9 with a guide RNA comprising the sequence as set forth in SEQ ID NO: 5, 6, 10, 12, 13 or 17.

8-12. (canceled)

13. The composition of claim 1, said composition further comprising at least one nucleic acid polymer consisting of SEQ ID NO:1 and SEQ ID NO: 4.

14. A method for the treatment of hepatitis B or hepatitis D infection comprising administering to a subject in need of treatment an effective amount of a pharmaceutically acceptable small molecule inhibiting the activity of casein kinase 1 isoform delta, DNAJB12, and microtubule-actin crosslinking factor 1.

15. The method of claim 14, wherein the small molecule is an oligonucleotide.

16. The method of claim 14, wherein the oligonucleotide is an antisense oligonucleotide, a synthetic interfering RNA, or a CRISPR-associated endonuclease and guide RNA complementary to any part of the mRNA for casein kinase 1 isoform delta, DnaJB12, casein kinase 1 isoform alpha, coatomer subunit epsilon, transducin beta-like protein 2, or microtubule-actin crosslinking factor 1.

17. The method of claim 14, wherein said composition inhibiting the assembly and/or secretion of HBV subviral particles (SVP).

18. The method of claim 14, wherein the small molecule is an antisense oligonucleotide with the sequence as set forth in SEQ ID NO: 12, 13 or 17.

19. The method of claim 14, wherein the small molecule is a synthetic interfering RNA with the sequence as set forth in SEQ ID NO: 5, 6, 10, 12, 13 or 17.

20. The method of claim 14, wherein the small molecule is CRISPR-Cas9 with a guide RNA comprising the sequence as set forth in SEQ ID NO: 5, 6, 10, 12, 13 or 17.

21. The method of claim 14, wherein said oligonucleotide comprises a modified nucleobase.

22. The method of claim 15, wherein said oligonucleotide is single stranded or double stranded.

23. The method of claim 15, wherein said oligonucleotide is a Speigelmer, an aptamer, an miRNA, a small interfering RNA (siRNA) or a small hairpin RNA (shRNA).

24. The method of claim 14, wherein said oligonucleotide comprises at least one modification in the phosphodiester linkage, on the sugar, and on the base.

25. The method of claim 14, wherein said oligonucleotide comprising at least one of a phosphorothioate linkage, a phosphorodithioate linkages, a 2′-O-methyl modification, a 2′-amino modification, a 2′-halo modification, an acyclic nucleotide analog, a 3′- and/or 5′-cap, 5′methylation of the cytosine base, and a 4′thioation of the uracil base.

26. The method of claim 14, further comprising administering at least one nucleic acid polymer consisting of SEQ ID NO:1 and SEQ ID NO: 4.

27. (canceled)


SEQUENCE LISTING (18 sequences)

SEQ ID NO: 1 - RNA, 40 nt, Artificial Sequence
  REP 2139, fully phosphorothioated, fully 2′ O methylribose modified, each cytosine 5′ methylated
acacacacacacacacacacacacacacacacacacacac

SEQ ID NO: 2 - RNA, 40 nt, Artificial Sequence; identical to SEQ ID NO: 1
  REP 2147, fully 2′ O methylribose modified, each cytosine 5′ methylated

SEQ ID NO: 3 - RNA, 20 nt, Artificial Sequence
  REP 2179, fully phosphorothioated, fully 2′ O methylribose modified, each cytosine 5′ methylated
acacacacacacacacacac

SEQ ID NO: 4 - RNA, 40 nt, Artificial Sequence; identical to SEQ ID NO: 1
  REP 2165, fully phosphorothioated, each cytosine 5′ methylated
  misc_feature 1-10, 12-20, 22-30, 32-40: 2′ O methylribose modification

SEQ ID NO: 5 - DNA, 18 bp, Artificial Sequence
  CSNK1D sense targeted sequence
aagagacagaaatacgaa

SEQ ID NO: 6 - DNA, 18 bp, Artificial Sequence
  DNAJB12 sense targeted sequence
caaggtgatggactgtat

SEQ ID NO: 7 - DNA, 18 bp, Artificial Sequence
  CopE sense targeted sequence
agctgttcgacgtaaaga

SEQ ID NO: 8 - DNA, 18 bp, Artificial Sequence
  CSNK1A1 sense targeted sequence
agaatttgcgatgtactt

SEQ ID NO: 9 - DNA, 18 bp, Artificial Sequence
  TBL2 sense targeted sequence
tgtcatcgacattggcat

SEQ ID NO: 10 - DNA, 18 bp, Artificial Sequence
  MACF1 sense targeted sequence
ccacaactaaaggaatta

SEQ ID NO: 11 - DNA, 18 bp, Artificial Sequence
  CopA sense targeted sequence
gtgagtacattgtgggtt

SEQ ID NO: 12 - DNA, 18 bp, Artificial Sequence
  CSNK1D antisense targeted sequence
ttcgtatttctgtctctt

SEQ ID NO: 13 - DNA, 17 bp, Artificial Sequence
  DNAJB12 antisense targeted sequence
atacagtccatcacctt

SEQ ID NO: 14 - DNA, 18 bp, Artificial Sequence
  CopE antisense targeted sequence
tctttacgtcgaacagct

SEQ ID NO: 15 - DNA, 18 bp, Artificial Sequence
  CSNK1A1 antisense targeted sequence
aagtacatcgcaaattct

SEQ ID NO: 16 - DNA, 18 bp, Artificial Sequence
  TBL2 antisense targeted sequence
aatgccaatgtcgatgac

SEQ ID NO: 17 - DNA, 18 bp, Artificial Sequence
  MACF1 antisense targeted sequence
ttaattcctttagttgtg

SEQ ID NO: 18 - DNA, 18 bp, Artificial Sequence
  CopA antisense targeted sequence
aaacccacaatgtactca